(19) United States
(12) Patent Application Publication
(10) Pub. No.: US 2026/0011007 A1
(43) Pub. Date: Jan. 8, 2026

(54) CARDIAC FIBROSIS DIAGNOSIS MODEL BASED ON MULTI-TASK ATTENTIONAL FEATURE FUSION

(71) Applicant: PLA General Hospital of Northern Theater Command, Shenyang City (CN)

(72) Inventors: Huishan Wang, Shenyang City (CN); Yueyang Wang, Shenyang City (CN); Yuji Zhang, Shenyang City (CN); Yongbo Song, Shenyang City (CN)

(21) Appl. No.: 19/052,646

(22) Filed: Feb. 13, 2025

(30) Foreign Application Priority Data
Jul. 4, 2024 (CN) 202410891964.3

(51) Int. Cl. G06T 7/00; A61B 5/00; A61B 5/055; G06V 10/26; G06V 10/72; G06V 10/764; G06V 10/82; G06V 20/70

(52) U.S. Cl. CPC G06T 7/0012; A61B 5/0044; A61B 5/055; G06V 10/26; G06V 10/72; G06V 10/764; G06V 10/82; G06V 20/70; G06T 2207/10088; G06T 2207/20081; G06T 2207/20084; G06T 2207/30048; G06V 2201/031

(57) ABSTRACT

The present application provides a cardiac fibrosis diagnosis model based on multi-task attentional feature fusion. The cardiac fibrosis diagnosis model is established by the following steps: S01: image collection and labeling: obtaining cardiac magnetic resonance (MR) images as sample data, and performing manual labeling to obtain heart labels corresponding to the MR images; S02: image preprocessing, including normalization processing, data enhancement, and data clipping; S03: model establishment, including establishment of an image recovery network and establishment of an image segmentation and classification network, and executing an image recovery task; S04: model pre-training: training the image recovery network such that the encoder of the image recovery network fully learns the feature of the cardiac fibrosis image; and S05: model training. An objective of the present application is to improve the segmentation precision and diagnosis accuracy of a network model for a cardiac fibrosis image.

### CROSS REFERENCE TO RELATED APPLICATION

This patent application claims the benefit and priority of Chinese Patent Application No. 202410891964.3, filed with the China National Intellectual Property Administration on Jul. 4, 2024, the disclosure of which is incorporated by reference herein in its entirety as part of the present application.

### TECHNICAL FIELD

The present application relates to the crossing technical field of deep learning and medical image recognition and diagnosis, and in particular, to a cardiac fibrosis diagnosis model based on multi-task attentional feature fusion.

### BACKGROUND

Cardiac fibrosis is an important pathological change which has a significant influence on the function of the heart. The accurate diagnosis of the cardiac fibrosis is of immense importance for the early detection and treatment of cardiovascular diseases. As shown in the World Health Report, the heat diseases are one of the main causes of death in the world, where the cardiovascular diseases (CVDs) cause about 17.3 million people to die per year. This number is predicted to exceed 23.6 million in 2030. The cardiac fibrosis, as a main histological feature of myocardial damage, is closely related to a plurality of heart diseases, such as arterial hypertension, valvular heart disease, diabetic cardiomyopathy, hypertrophic cardiomyopathy, dilated cardiomyopathy, and cardiac aging, etc. Cardiac Magnetic Resonance (CMR) imaging is an important means for diagnosing the cardiac fibrosis, which can effectively identify the cardiac fibrosis especially by Late Gadolinium Enhancement (LGE). The CMR imaging is to create a detailed image of the heart and its surroundings by using a strong magnetic field and a computer, and thus plays an important role in detecting and tracking the congenital heart disease or the acquired heart disease. However, the accurate diagnosis of the cardiac fibrosis relies on the high spatial resolution imaging technology. The traditional diagnosis methods might need to manually extract features, and thus are time-consuming and laborious and may easily have errors. Therefore, the development of an automatic diagnosis method is of great value for improving the diagnosis efficiency and reducing errors.

At present, the automatic diagnosis of the cardiac fibrosis has achieved a lot of work, where methods may be approximately divided into two types: a classical machine learning algorithm, and automatic diagnosis using deep learning. The work on the machine learning algorithm is as follows: Pu et al. collected the images of 273 cardiomyopathy patients for radiomics research. Predictive influence features were found by using logistic regression analysis, and a CMR model was established. Radiomics features are extracted from the maximum wall thickness (MWT) level and the whole left ventricular (LV) myocardium. A radiomics model was established by extreme gradient boosting. A comprehensive model was established by fusing image features and the radiomics model, which achieved the final diagnostic accurate rate of 89.02%, the sensitivity of 92.54%, and the AUC value of 0.898. Campese et al. combined the support vector machine (SVM) with the convolutional neural network (CNN) to complete the binary task of determining whether the heart tissue has scars. The final model achieved the accuracy of 71% and the sensitivity of 72%.

The work on the use of the deep learning algorithm is as follows: Popescu et al. developed a multi-stage network based on deep learning to automatically segment the myocardium and the scar fibrosis in Contrast Enhancement Cardiac Magnetic Resonance (CE-CMR) imaging, and extract clinical features. Specifically, a three-stage neural network was established. Firstly, a left ventricular (LV) region of interest (ROI) was identified, and then the ROI was segmented as an active myocardium and enhancement region, and finally, a prediction result was adjusted through a post-processing stage to conform to an anatomical constraint. In total, 155 two-dimensional CE-CMR patient scans and 246 synthetic LGE sample scans were used for training and testing. The results showed that the predicted left ventricle segmentation and the scar segmentation achieved the balanced accuracy of 96% and the balanced accuracy of 75%, respectively. Marco et al. established a convolutional neural network model to solve the problem of the detection of the myocardial fibrosis in an early Contrast Enhancement Cardiac Computed Tomography (CE-CCT) image. CE-CMR and (early and late) CE-CCT examinations were conducted on 50 patients known with left ventricular dysfunction (LVD). According to the CE-CMR mode, the patients were classified as ischemic or non-ischemic LVD. The researchers extracted myocardial segments on the early CE-CCT image according to the 16-segment model of the American Heart Association (AHA) and labeled them as having scars or no scar based on the manual tracking of the late CE-CCT. The developed deep learning model was employed to classify each segment. By analyzing 44,187 left ventricular segments, the model achieved the accuracy rate of 71% and the AUC of 76%. Moreover, by comparing the CE-CMR result and the corresponding early CE-CCT result, the consistency of the model and the reality reached 89%. This indicated that the left ventricular segments affected by the myocardial fibrosis were detected in early CE-CCT acquisition by deep learning without extra contrast medium injection or radiation dosage.

The cardiac fibrosis is a pathological state characterized by abnormal deposition of the non-contractile extracellular matrix in the cardiac mesenchyme, and the abnormal deposition results in changes in cardiac structure and function. The characteristics of the cardiac fibrosis include the hardening of the heart tissue and the formation of the scars. These changes might result in systolic and diastolic dysfunctions. At present, the process of diagnosing the cardiac fibrosis has the following problems:
1. Limitations of the machine learning algorithm: there are limitations on the use of the machine learning algorithm to diagnose the cardiac fibrosis. Machine learning highly relies on manual feature engineering, and requires a medical expert to extract features from a plurality of data sources such as a lot of medical images, a blood examination indicator, and a biomarker level. Different experts may have different opinions on which features are the most important. This leads to a consistency problem in the model training process, thereby affecting the stability and accuracy of model diagnosis.
2. The cardiac fibrosis data has heterogeneity: the clinical manifestations and the imaging features of the cardiac fibrosis patients might vary due to individual differences and different disease stages and complications, leading to high data heterogeneity. When the machine learning algorithm and the simple neural network algorithm process this type of data, it might be difficult to capture the core pathological features of the cardiac fibrosis disease, thereby affecting the final diagnosis accuracy on diseases.
3. There is little cardiac fibrosis data: in the current research work, there are relatively few samples of the cardiac fibrosis patients. Usually, there is only data of hundreds of cases. For machine learning or deep learning, generalized features cannot be extracted. The extraction capability of the model is insufficient, which finally leads to a reduced diagnosis accuracy rate of the cardiac fibrosis disease.
4. ROI labeling is required: for most existing deep learning algorithms, the heart parts in MR images needs to be labeled first, and then the neural network is allowed to learn the corresponding heat labeled images to complete the diagnosis of the cardiac fibrosis. This approach is not end-to-end extraction and needs to manually label the heart. Making the heat each time needs to take extra time.
5. A diseased area has an unclear boundary: the cardiac fibrosis may manifest as focal or diffuse, and its morphological features might include a banding, spotty, or sheet distribution. Different types of fibrosis have different forms. Therefore, the boundaries of different forms of areas from the normal myocardium may also be different.

Regarding the above problems, the present application provides a cardiac fibrosis diagnosis model based on multi-task attentional feature fusion.

Regarding the problem 1 and the problem 2, the present application proposes an improved Mask Region-based Convolutional Neural Network (Mask R-CNN) algorithm, in which an innovative dual-level feature pyramid network structure is introduced, including a pixel-feature pyramid network (p-FPN) and a region-feature pyramid network (r-FPN) to realize more elaborate feature fusion and a gradually increasing mask resolution. Moreover, in the present application, an efficient feature aggregation module (FAM) is further designed. By dynamically adjusting region-feature and pixel-feature weights, learning the key features of the cardiac fibrosis image by the model is enhanced. Meanwhile, with a self-attention feature fusion mechanism, the segmentation precision and the diagnosis accuracy of the network model for the cardiac fibrosis task are improved.

Regarding the problem 3, the present application adopts the self-supervised learning technology to enhance the extraction capability of the model for the features in the cardiac MR images. In particular, an encoder-decoder architecture is designed in the present application. The architecture uses a ResNet module as the basis of the encoder. Next, random masking is performed on an original image. The objective of image recovery is to reconstruct a mask part in a reconstructed image. Meanwhile, the randomly masked MR image is randomly zoomed in or out. Random rotation is used as data enhancement. The model then recovers the original cardiac MR image through two stages of feature extraction and image reconstruction. Through this process, the encoder can deeply mine the intrinsic semantic information of the cardiac fibrosis data. The dependence on a large-scale labeled data set is reduced, and the problem of few samples of cardiac fibrosis patients is ameliorated.

Regarding the problem 4, the present application proposes the improved Mask R-CNN algorithm. Cardiac MR images are segmented and classified by the model. The generalization capability of the model can be improved by multi-task learning. Meanwhile, this is an end-to-end network structure. The traditional manual labeling process can be simplified. With the advanced functions of the network model, the algorithm of the present application can automatically identify the segmented heart region and diagnose the cardiac fibrosis.

Regarding the problem 5, the present application adopts a combined strategy of a plurality of loss functions in the neural network training process. These loss functions include a classification loss, a bounding box loss, a dice loss, and an edge loss specially designed for the unclear boundary problem. A calculation method of the edge loss is as follows: firstly, a laplacian operator is applied to a real mask image to enhance an edge feature in the image. An enhanced soft edge map is then converted to a clear binary edge map by thresholding processing. Likewise, a mask image predicted by the model is subjected to edge detection and binarization steps to generate a corresponding predicted edge map. Finally, a difference between the predicted edge map and the real edge map is calculated to obtain the edge loss. The edge feature of the cardiac fibrosis is emphasized by the laplacian operator, helping the model to capture the high-frequency detail information of the cardiac fibrosis form.

### SUMMARY

An objective of the present application is to improve the segmentation precision and the diagnosis accuracy of a network model for a cardiac fibrosis image.

In order to achieve the above objective, the present application provides the following basic solutions.

A cardiac fibrosis diagnosis model based on multi-task attentional feature fusion is provided.

The principles and the effects of the basic solution are as follows:
1. Compared with the prior art, an improved Mask R-CNN algorithm is used for a network. In the present application, an innovative dual-level feature pyramid network structure is introduced, including a pixel-FPN and a region-FPN. An efficient feature aggregation module (FAM) is further defined.
2. An encoder-decoder architecture is established for self-supervised learning. The encoder part uses a ResNet module as the basis. An input to a model is an original image which is subjected to random masking processing. An objective of the model is to reconstruct a part occluded by a mask in an image. Meanwhile, we implemented a plurality of data enhancement techniques for a randomly masked MR image. A trained encoder model is used as a Backbone and integrated into the improved Mask R-CNN algorithm.
3. The present application uses the improved Mask R-CNN algorithm. A cardiac MR image is segmented and classified by the model. This is multi-task learning for the model. The generalization capability of the model can be improved. Meanwhile, the problem of needing manual labeling is solved. An end-to-end network structure design is achieved.
4. A plurality of loss functions are used, including a classification loss, a bounding box loss, a dice loss, and a proposed edge loss.
5. Compared with the prior art, in the prior art, for a cardiac fibrosis diagnosis task, machine learning dependent manual feature engineering is mostly used or a simple convolutional neural network is used for identification and diagnosis. This approach may lead to a long time and a low accuracy rate. The present application proposes an improved Mask R-CNN algorithm, where a dual-level feature pyramid network structure (Dual-Level FPN) is used, including a pixel-FPN and a region-FPN, which can capture a global context and local details of the cardiac MR image to realize more elaborate feature fusion. The designed FAM enhances the learning of the key features of a cardiac fibrosis image by the model by dynamically adjusting a region-feature weight and a pixel-feature weight. Meanwhile, with a self-attention feature fusion mechanism, the segmentation precision and the diagnosis accuracy of the network model for the cardiac fibrosis image are improved.
6. In the prior art, there are few samples of cardiac fibrosis patients (only hundreds of cases), which limits the generalization capability of the model. In order to solve the problem, the present application adopts the self-supervised learning technology to enhance the extraction capability of the model for a feature of a cardiac MR image. We designed a model based on the encoder-decoder architecture, where the encoder part uses the ResNet module as the basis. An input to the model is an original image which is subjected to random masking processing. An objective of the model is to reconstruct a part occluded by a mask in an image. In order to further enhance the robustness of the model, we implemented a plurality of data enhancement techniques for a randomly masked MR image. Through the two stages of feature extraction and image reconstruction, the model can recover an original cardiac MR image. This process urges the encoder to deeply mine the intrinsic semantic information of the cardiac fibrosis data, thereby reducing the dependence on a large-scale labeled data set and effectively ameliorating the problem of insufficient samples. We used the encoder model as the Backbone, which is integrated into the improved Mask R-CNN algorithm. This integration enables the model to locate and identify a diseased area more accurately when processing a cardiac fibrosis MR image, thereby improving the accuracy and reliability of diagnosis.
7. In the prior art, the heart part in an MR image needs to be marked first, and then the neural network is allowed to learn the corresponding heart marked image to complete the diagnosis of the cardiac fibrosis. The present application uses the improved Mask R-CNN algorithm with a multi-task learning framework. Thus, not only is the identification capability of the model to the heart region enhanced, but also the generalization performance of the model is improved. This multi-task learning strategy enables the network to learn both segmentation and classification features in the cardiac fibrosis task, thereby sharing knowledge between different tasks. The overall diagnosis accuracy is improved. This algorithm adopts the end-to-end network structure design. This means that the whole process from inputting an original MR image to final cardiac segmentation and classification outputting is automated without manual intervention. Such an end-to-end design significantly reduces the dependence on manual labeling, thereby saving valuable time resources and also reducing errors introduced by manual operations.
8. In the prior art, a model cannot accurately distinguish cardiac fibrosis edges. The present application adopts a combined strategy of a plurality of loss functions in the neural network training process. These loss functions include a classification loss, a bounding box loss, a dice loss, and an edge loss specially designed for the unclear boundary problem. The edge loss highlights the edge feature of the cardiac fibrosis region by using the laplacian operator, thereby helping the model to capture the high-frequency detail information of the cardiac fibrosis form more accurately. Such an edge loss design enables the model to better understand and distinguish the boundaries between different types of cardiac fibrosis regions and the normal myocardium. By the training method with a combination of a plurality of loss functions, the model of the present application can identify and locate the cardiac fibrosis diseased area more accurately, thereby improving the accuracy and reliability of cardiac fibrosis diagnosis.

Further, in step S01, the cardiac MR image includes two different labels: No. 0 label and No. 1 label; the No. 0 label is a background label, and the No. 1 label is the heart label; whether the heart has fibrosis is determined, and marked with 0 and 1, 0 representing normal and 1 representing fibrosis; and the labeled sample data is divided into a training set and a test set.

further, in step S02, the normalization processing is performed according to (x−μ)/σ, where x represents a hounsfield unit (HU) value of a pixel in a cardiac MR image; u represents an average value of HU values of all pixels; σ represents a standard deviation of all the pixels; and for the data enhancement, Gaussian random noise, random contrast enhancement, random mirroring, random horizontal flipping, and random rotation are used.

Further, in step S03, the feature of the cardiac fibrosis image includes texture and edge information; the convolutional block is specifically composed of a convolutional layer, a LeakyReLU activation function and a BN layer; the convolutional layer is configured to further extract an advanced feature of the cardiac MR image; the LeakyReLU activation function is configured to introduce a nonlinear change such that the cardiac fibrosis diagnosis model is capable of learning and simulating more complicated function mapping; and the BN layer is configured to increase a training speed.

Further, in step S03, the image segmentation and classification network is configured to further refine and upsample a feature map extracted by the encoder to recover a resolution close to a resolution of an original input image; and the ConvTranspose layer is configured to reduce artifacts and blurs in an image using structural information in training data.

Further, in step S03, an overall network structure is improved based on the Mask R-CNN; a first improvement is made on the BackBone of the image segmentation and classification network, which is replaced with an encoding layer of the image recovery network in step S03, and a parameter of the encoding layer is migrated; a cardiac image recovery task is completed by the image recovery network through self-supervised learning; semantic information and a typical feature of the cardiac image are learned by the encoding layer; a network parameter is migrated, and a convergence rate of the improved Mask R-CNN model is increased; a dual-level feature pyramid network structure is introduced into the FPN, and includes a pixel-FPN and a region-FPN to capture a global context and local details of the cardiac MR image; and the FAM is configured to dynamically adjust a region-feature weight and a pixel-feature weight.

Further, in step S04, in the model pre-training, an Adam optimizer is adopted for the model training, with a specific formula being as follows:

$$MSE = \frac{1}{N} {\sum }_{i = 1}^{N} {( {y}_{i} - {\hat{y}}_{i} )}^{2}$$

where N represents a total number of samples; yᵢ represents a real value of an ith sample; and ŷᵢ represents a predicted value of the ith sample.

Further, in step S05, a patience parameter is set for 50 times; when Loss does not decrease in 50 consecutive Epochs, a learning rate automatically decreases to 1/10 of an original learning rate; 5000 rounds of training are performed; a loss function uses a combination of a plurality of losses, including a dice loss, a cross entropy loss, and an edge loss proposed for a problem of an unclear boundary of a cardiac fibrosis diseased area; and a formula of the dice loss is as follows:

$${L}_{Dice} = 1 - \frac{2 \times \vert T ⋂ P \vert}{\vert T \vert + \vert P \vert}$$

where T represents true labeling; P represents a predicted region; and the dice loss function is configured to maximize an intersection of a model output and a real label.

Further, in step S05, a formula of the cross entropy loss is as follows:

$${L}_{ce} = - [ y log ( \hat{y} ) + ( 1 - y ) log ( 1 - \hat{y} ) ]$$

where y represents the real label, and ŷ represents a prediction probability of 0 to 1.

Further. a calculation method of the edge loss designed for the problem of an unclear cardiac fibrosis boundary includes:
a first step: generating an edge map, and using a laplacian operator to operate a real mask ground-truth mask to obtain a soft edge map reflecting edge information, where the laplacian operator is a second order derivative operator and is capable of highlighting an edge and a detail in an image; and specific steps are as follows:
1, defining a laplacian kernel, where the kernel used is as follows:

$$K = [ \begin{matrix}0 & 1 & 0 \\ 1 & - 5 & 1 \\ 0 & 1 & 0\end{matrix} ]$$

2, a convolution operation: performing a convolution operation using the laplacian kernel and the real mask; and for each pixel in the real mask, multiplying a pixel in a neighborhood thereof by a corresponding value in the laplacian kernel, and then summating results to obtain a new value of the pixel point; and
3, result conversion, superposing an image result after processing with a laplacian filter kernel and an original image to enhance an edge while retaining an image edge content;
a second step: performing thresholding processing on the soft edge map obtained through the laplacian operator, and converting the soft edge map to a binary edge map, where in the binaryzation process, pixels in the edge map are divided into two an edge type and a non-edge type;
a third step: predicting an edge map: repeating the first step and the second step with a mask predicted by a model to obtain a predicted edge map; and
a fourth step: calculating an edge loss: calculating the edge loss using the formula of the dice loss, thereby obtaining a training loss function Lₜₒₜₐₗ: Lₜₒₜₐₗ=L_{Dice}+L_{ce}+Ledge.

### BRIEF DESCRIPTION OF THE DRAWINGS

To describe the technical solutions in the embodiments of the present application more clearly, the drawings required to describe the embodiments are briefly described below. Apparently, the drawings described below are only some embodiments of the present application. Those of ordinary skill in the art may further obtain other drawings based on these drawings without creative efforts.

FIG. 1 illustrates a schematic diagram of an image recovery network in a cardiac fibrosis diagnosis model based on multi-task attentional feature fusion provided by an embodiment of the present application;

FIG. 2 illustrates a specific constitutional diagram of a convolutional block in a cardiac fibrosis diagnosis model based on multi-task attentional feature fusion provided by an embodiment of the present application;

FIG. 3 illustrates a schematic diagram of an image segmentation and classification network in a cardiac fibrosis diagnosis model based on multi-task attentional feature fusion provided by an embodiment of the present application;

FIG. 4 illustrates a process diagram of a pixel-FPN in a cardiac fibrosis diagnosis model based on multi-task attentional feature fusion provided by an embodiment of the present application;

FIG. 5 illustrates a schematic diagram of a structural design of a region-FPN in a cardiac fibrosis diagnosis model based on multi-task attentional feature fusion provided by an embodiment of the present application; and

FIG. 6 illustrates a schematic diagram of a feature aggregation module (FAM) in a cardiac fibrosis diagnosis model based on multi-task attentional feature fusion provided by an embodiment of the present application.

### DETAILED DESCRIPTION OF THE EMBODIMENTS

To further describe the technical means adopted by the present application to achieve the intended purpose and the effects of the technical means, the specific implementations, structures, features, and effects of the present application are described in detail below with reference to the drawings and preferred embodiments.

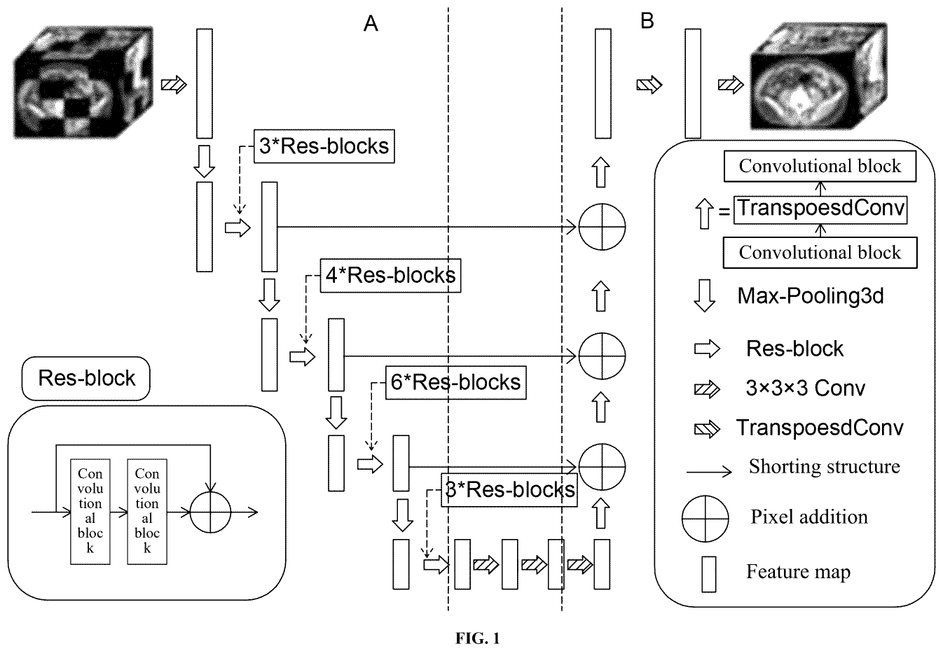

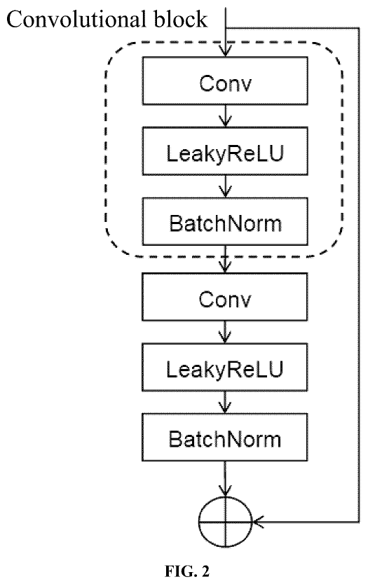

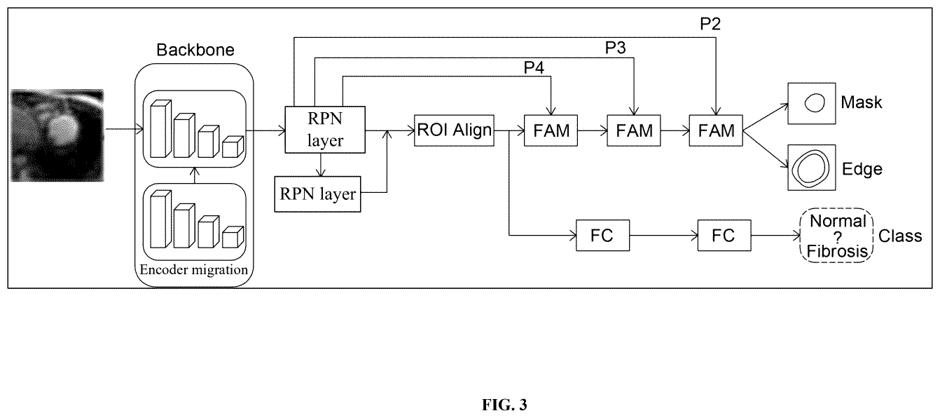

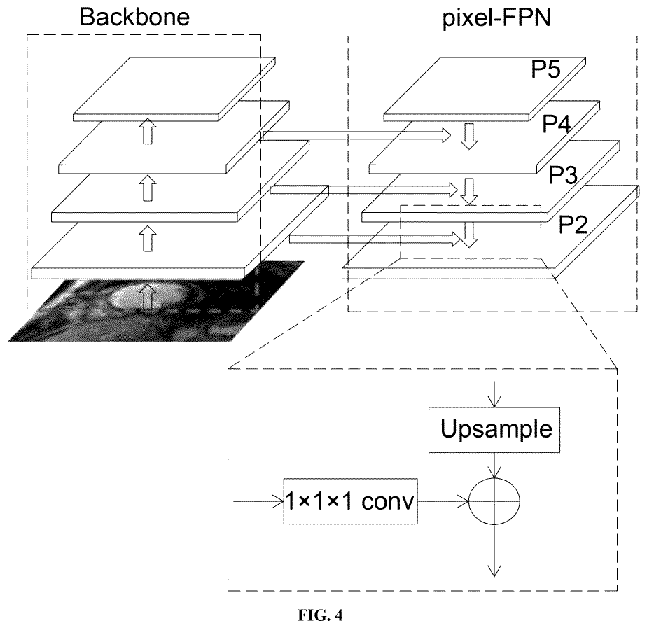

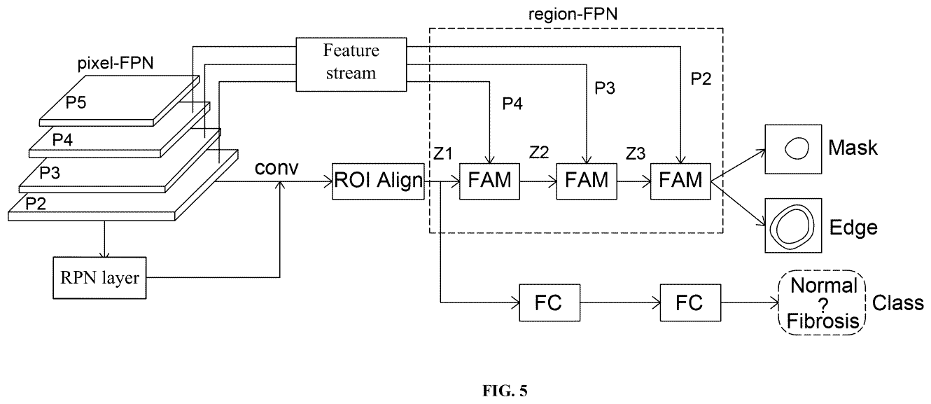

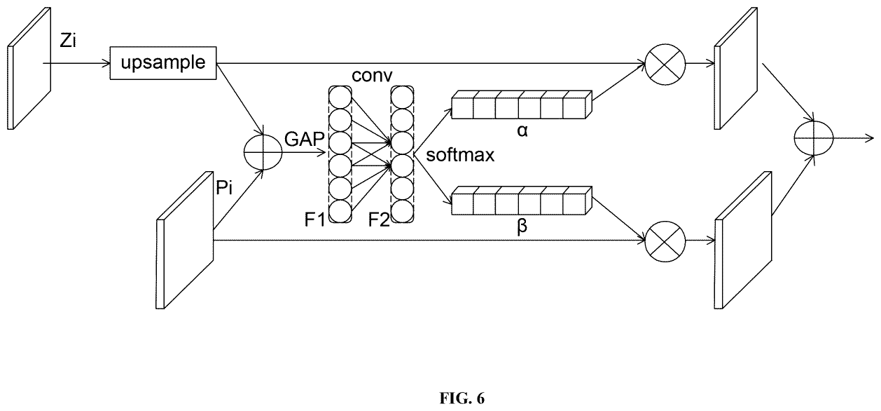

The embodiments are as shown in FIG. 1, FIG. 2, FIG. 3, FIG. 4, FIG. 5, and FIG. 6.

A cardiac fibrosis diagnosis model based on multi-task attentional feature fusion is provided.

The cardiac fibrosis diagnosis model is established by the following steps.

S01: image collection and labeling: cardiac MR images are obtained as sample data, and manual labeling is performed to obtain heart labels corresponding to the MR images.

Specifically, the cardiac MR images are obtained as the sample data and manually labeled to obtain the heart labels corresponding to the MR images. The cardiac MR image includes 2 different labels: No. 0 label and No. 1 label. The No. 0 label is a background label, and the No. 1 label is the heart label. Meanwhile, whether the heart has fibrosis is determined, and marked with 0 and 1, 0 representing normal and 1 representing fibrosis. The labeled sample data is divided into a training set and a test set. Each of the training set and the test set has two labels: the No. 0 label is the background label, and the No. 1 label is the heart label.

S02: image preprocessing: the image preprocessing includes normalization processing, data enhancement, and data clipping; and the images are preprocessed by the normalization processing, the data enhancement, and the data clipping.

Specifically, in step S02, the normalization processing is performed according to (x−μ)/σ, where x represents a hounsfield unit (HU) value of a pixel in a cardiac MR image; u represents an average value of HU values of all pixels; σ represents a standard deviation of all the pixels; for the data enhancement, Gaussian random noise, random contrast enhancement, random mirroring, random horizontal flipping, and random rotation are used; and for the data clipping, the data is clipped with a clipping size [128,128,128].

S03: model establishment: the model establishment includes establishment of an image recovery network and establishment of an image segmentation and classification network, where the image recovery network is configured to execute an image recovery task by self-supervised learning; and the image segmentation and classification network is established to segment a cardiac image and diagnose cardiac fibrosis.

The model establishment is specifically as follows:

The image recovery network includes an encoder and a decoder. The encoder is a part A, and includes a convolutional layer, a Res-Block, and a pooling layer.

The convolutional layer is configured to extract a feature of a cardiac fibrosis image.

The Res-Block is composed of two convolutional blocks, and enables the image recovery network to learn identity mapping more easily.

The pooling layer is configured to highlight a significant feature in the cardiac fibrosis image and reduce a calculation quantity and a number of parameters.

The image recovery network is specifically described below.

The image recovery network is composed of encoder and decoder structures. The encoder is a part A, and includes a convolutional layer, a Res-Block, and a pooling layer. Firstly, the encoder part is described. The convolutional layer uses 3×3×3 convolutions, and is configured to extract a feature of a cardiac fibrosis image. The pooling layer is configured to highlight a significant feature in the cardiac fibrosis image and reduce a calculation quantity and a number of parameters. Finally, the Res-Block is composed of two convolutional blocks. The specific composition of the convolutional block is as shown in FIG. 2. The convolutional layer is configured to further extract an advanced feature of the cardiac MR image; the LeakyReLU activation function is configured to introduce a nonlinear change such that the cardiac fibrosis diagnosis model is capable of learning and simulating more complicated function mapping; and the BN layer is configured to increase a training speed. Model generalization is improved. An activation output is adjusted and zoomed such that an input distribution of each layer is more stable. The internal covariant shift problem is reduced. The shorting structure enables the image recovery network to learn identity mapping more easily. Thus, the degeneration problem in a deep network is solved.

The decoder is a part B, and includes an upsampling layer, a convolutional block, and a shorting structure.

The upsampling layer is configured to upsample a low-resolution feature map to a higher resolution by using a ConvTranspose layer.

The convolutional block is identical in composition to the convolutional block used in the establishment of the image recovery network.

The shorting structure is configured to directly connect a low-level feature in the encoder to a corresponding layer of the decoder.

The decoder is a part B, and includes an upsampling layer, a convolutional block, and a shorting structure, and is configured to further refine and upsample a feature map extracted by the encoder to recover a resolution close to a resolution of an original input image. The composition of the convolutional block is the same as above. Upsampling is performed by using a ConvTranspose layer, which is a transposed convolution operation and is configured to upsample a low resolution feature map to a higher resolution by learning. Compared with the traditional upsampling method (e.g., nearest neighbor interpolation), the ConvTranspose layer can learn the feature upsampling process more effectively. Since the ConvTranspose layer utilizes structural information in training data, for an image recovery task, it can reduce artifacts and blurs in an image. The shorting structure is configured to directly connect a low-level feature in the encoder to a corresponding layer of the decoder so that the image recovery network can learn identity mapping more easily. The vanishing gradient problem in the deep network is solved. The image recovery network is further allowed to retain low-level fine features in the upsampling process, which is very effective for accurately recovering the cardiac fibrosis image. The shorting structure further enhances the representation capability of the image recovery network so that the image recovery network can learn both global and local features, thereby further enhancing the recovery effect.

The establishment of the image segmentation and classification network including making an improvement based on a Mask R-CNN, and a Backbone of the image segmentation and classification network, a feature pyramid network (FPN), and a feature aggregation module (FAM) are established.

Specifically, as shown in FIG. 3, an overall network structure is improved based on the Mask R-CNN; a first improvement is made on the BackBone of the image segmentation and classification network, which is replaced with an encoding layer of the image recovery network in the first stage, and a parameter of the encoding layer is migrated; a cardiac image recovery task is completed by the image recovery network through self-supervised learning; semantic information and a typical feature of the cardiac image are learned by the encoding layer; a network parameter is migrated, and a convergence rate of the improved Mask R-CNN model can be increased, and the feature extraction capability of the model for a cardiac fibrosis is improved. A second improvement is made on the FPN. The present application introduces a dual-level feature pyramid network structure, including a pixel-FPN and a region-FPN, which can capture a global context and local details of the cardiac MR image. More elaborate feature fusion is realized. A third improvement is made on the designed FAM. By dynamically adjusting a region-feature weight and a pixel-feature weight, learning the key features of the cardiac fibrosis image by the model is enhanced. Meanwhile, with a self-attention feature fusion mechanism, the segmentation precision and the diagnosis accuracy of the network model for the cardiac fibrosis image are improved.

As shown in FIG. 4, there is shown the process of the pixel-FPN. The pixel-FPN is of a top-down network structure, which is configured to capture the context information in an image through feature maps of a plurality of scales. These feature maps are from different levels of the encoding layer in the image recovery network. Each level corresponds to different resolutions. Thus, a multi-scale feature representation is formed. By integrating the semantic information from different levels of the network, the model is helped to understand the image content on different scales. The top-down structural design enables the semantic information of a higher level to be effectively transferred to a lower level. The understanding of the global context by the model can be enhanced. Meanwhile, the established feature pyramid reuses the same feature at a plurality of resolutions. The computing efficiency is improved.

As shown in FIG. 5,
the figure shows the structural design of the region-FPN. The region-FPN, which is a new structure proposed in the present application, is configured to generate top-down region feature levels by gradually fusing complementary information from the pixel-FPN starting from a region feature aligned with the ROI. The spatial resolutions of these features are gradually increased with the increasing of the levels. The region-FPN focuses on enhancing the region features so that the model can capture more granular visual information of a cardiac fibrosis image, especially on edges and details of a target. The FAM utilizes the self-attention fusion mechanism to improve effective fusion of a region-feature and a pixel-feature. Region Proposal Network (RPN) plays a same role as in the Mask R-CNN network. A high-quality candidate region is generated rapidly on a feature map so that the network can effectively detect targets of various sizes. ROI Align network plays a role in mapping the candidate region ROI generated by the RPN to a feature map of a fixed size to facilitate accurate target classification and mask generation. It needs to be noted that we used the Mask R-CNN of the 3D version. Here, the ROI Align network selects trilinear interpolation to interpolate a feature in three dimensions, but its function is identical to that of the ROI Align network in the classical Mask R-CNN. The candidate region generated by the RPN is accurately mapped to the feature map of the fixed size through the ROI Align network for subsequent segmentation and classification. For classification, the feature map is subsequently expanded and then connected with two fully connected layers FC for determination.

As shown in FIG. 6,
the FAM is composed of an upsampling layer, a convolutional layer, a global average pooling (GAP) module, and a softmax module. Zi represents an ROI aligned feature, i.e., a pixel-FPN feature. Pi represents a region-FPN feature. The FAM firstly upsamples the feature Zi such that it is consistent with the feature Pi in size, and then adds up 2 features for fusion. GAP is performed on the added feature map to generate a channel feature vector F1. One-dimensional convolution is performed on the channel feature vector. Information interaction between channels is performed to obtain a feature vector F2, and then channel weighting is performed on the feature vector using softmax. Weight vectors α and β corresponding to the feature Zi and the feature Pi are calculated. The weight vectors are separately multiplied by the feature map to obtain an attention enhanced feature map of Zi and an attention enhanced feature map of Pi. Finally, the two enhanced feature maps are added up to obtain a mixed enhanced feature map. The FAM enhances the learning of the key features of cardiac images by the model. Meanwhile, based on the self-attention feature fusion mechanism, the model is helped to better learn the mixed features of the cardiac images. The segmentation precision of the network model for a cardiac fibrosis task is improved.

S04: model pre-training: the image recovery network is trained such that the encoder of the image recovery network fully learns the feature of the cardiac fibrosis image.

Specifically, in step S04, the image recovery network is trained such that the encoder of the network fully learns the feature of the cardiac fibrosis image. In the model training, an Adam optimizer is adopted for the model training. Momentum parameters are set to 0.93 and 0.99, and an initial learning rate is set to 0.0001:5000 rounds of training are performed such that the randomly occluded part in an input image is repaired. The loss function is a mean square error (MSE) loss function, which is configured to measure a mean value of squares of a difference between a predicted value of the model and an actual value. A specific formula is as follows:

$$MSE = \frac{1}{N} {\sum }_{i = 1}^{N} {( {y}_{i} - {\hat{y}}_{i} )}^{2}$$

where N represents a total number of samples; yᵢ represents a real value of an ith sample; and ŷᵢ represents a predicted value of the ith sample.

S05: model training: after completion of the model pre-training, the encoder of the image recovery network is used as the BackBone of the Mask R-CNN, and an encoder parameter trained at a first stage is migrated to the Mask R-CNN, and model training is performed by automatically decreasing the learning rate using ReduceLROnPlateau.

The model training is specifically as follows:

After completion of the model pre-training, the encoder of the resulting image recovery network is used as the BackBone in the Mask R-CNN, and the encoder parameter trained at the first stage is migrated to the Mask R-CNN. The initial learning rate is set to 0.0001, and automatic learning rate decreasing of ReduceLROnPlateau is adopted. A patience parameter is set for 50 times. When Loss does not decrease in 50 consecutive Epochs, the learning rate automatically decreases to 1/10 of an original learning rate. 5000 rounds of training are performed. A loss function uses a combination of a plurality of losses, including a dice loss, a cross entropy loss, and an edge loss proposed for a problem of an unclear boundary of a cardiac fibrosis diseased area.

A formula of the dice loss is as follows:

$${L}_{Dice} = 1 - \frac{2 \times \vert T ⋂ P \vert}{\vert T \vert + \vert P \vert}$$

where T represents true labeling; P represents a predicted region; and the dice loss function is configured to maximize an intersection of a model output and a real label.

In step S05, a formula of the Cross entropy is as follows:

$${L}_{ce} = - [ y log ( \hat{y} ) + ( 1 - y ) log ( 1 - \hat{y} ) ]$$

where y represents the real label, and ŷ represents a prediction probability of 0 to 1.

For the problem of an unclear cardiac fibrosis boundary, the edge loss is designed in the present application.

The calculation method includes the following steps.

In a first step, an edge map is generated, and a laplacian operator is used to operate a real mask ground-truth mask to obtain a soft edge map reflecting edge information, where the laplacian operator is a second order derivative operator and is capable of highlighting an edge and a detail in an image; and specific steps are as follows:
1, a laplacian kernel is defined, where the kernel used is as follows:

$$K = [ \begin{matrix}0 & 1 & 0 \\ 1 & - 5 & 1 \\ 0 & 1 & 0\end{matrix} ]$$

2, a convolution operation: a convolution operation is performed using the laplacian kernel and the real mask; and for each pixel in the real mask, a pixel in a neighborhood thereof is multiplied by a corresponding value in the laplacian kernel, and then results are summated to obtain a new value of the pixel point; and
3, result conversion, an image result after processing with a laplacian filter kernel and an original image are superposed to enhance an edge while retaining an image edge content.

In a second step, thresholding processing is performed on the soft edge map obtained through the laplacian operator, and the soft edge map is converted to a binary edge map, where in the binaryzation process, pixels in the edge map are divided into two an edge type and a non-edge type.

In a third step, an edge map is predicted; the steps are repeated with a mask predicted by a model to obtain a predicted edge map.

In a fourth step, an edge loss is calculated; the edge loss is calculated using the formula of the dice loss, thereby obtaining a training loss function Lₜₒₜₐₗ: Lₜₒₜₐₗ=L_{Dice}+L_{ce}+Ledge.

Key points of the present application:
1. An improved Mask R-CNN algorithm is used for a network. In the present application, an innovative dual-level feature pyramid network structure is introduced, including a pixel-FPN and a region-FPN. An efficient feature aggregation module (FAM) is further defined.
2. An encoder-decoder architecture is established for self-supervised learning. The encoder part uses a ResNet module as the basis. An input to a model is an original image which is subjected to random masking processing. An objective of the model is to reconstruct a part occluded by a mask in an image. Meanwhile, we implemented a plurality of data enhancement techniques for a randomly masked MR image. A trained encoder model is used as a backbone and integrated into the improved Mask R-CNN algorithm.
3. The present application uses the improved Mask R-CNN algorithm. A cardiac MR image is segmented and classified by the model. This is multi-task learning for the model. The generalization capability of the model can be improved. Meanwhile, the problem of needing manual labeling is solved. An end-to-end network structure design is achieved.
4. A plurality of loss functions are used, including a classification loss, a bounding box loss, a dice loss, and a proposed edge loss.

The above embodiments are only preferred embodiments of the present application, and are not intended to limit the present application in any form. Although the present application is disclosed through the above preferred embodiments, these preferred embodiments are not intended to limit the present application. Any person skilled in the art may make some changes or modifications to the above technical contents without departing from the scope of the technical solution of the present application. However, such changes or modifications should be deemed as equivalent embodiments of the present application. Any simple modification, equivalent change and modification made to the above embodiments according to the technical essence of the present application without departing from the content of the technical solution of the present application should fall within the scope of the technical solution of the present application.

What is claimed is:

1. A cardiac fibrosis diagnosis model based on multi-task attentional feature fusion, established by the following steps:
S01: image collection and labeling: obtaining cardiac magnetic resonance (MR) images as sample data, and performing manual labeling to obtain heart labels corresponding to the MR images;
S02: image preprocessing, comprising normalization processing, data enhancement, and data clipping: preprocessing the images by the normalization processing, the data enhancement, and the data clipping;
S03: model establishment, comprising establishment of an image recovery network and establishment of an image segmentation and classification network, wherein the image recovery network is configured to execute an image recovery task by self-supervised learning; and the image segmentation and classification network is established to segment a cardiac image and diagnose cardiac fibrosis;
wherein:
the image recovery network comprises an encoder and a decoder; the encoder is a part A, and comprises a convolutional layer, a Res-Block, and a pooling layer;
the convolutional layer is configured to extract a feature of a cardiac fibrosis image;
the Res-Block is composed of two convolutional blocks, and enables the image recovery network to learn identity mapping more easily;
the pooling layer is configured to highlight a significant feature in the cardiac fibrosis image and reduce a calculation quantity and a number of parameters;
the decoder is a part B, and comprises an upsampling layer, a convolutional block, and a shorting structure;
the upsampling layer is configured to upsample a low-resolution feature map to a higher resolution by using a ConvTranspose layer;
the convolutional block is identical in composition to the convolutional block used in the establishment of the image recovery network;
the shorting structure is configured to directly connect a low-level feature in the encoder to a corresponding layer of the decoder; and
the establishment of the image segmentation and classification network comprises making an improvement based on a Mask Region-based Convolutional Neural Network (Mask R-CNN), and establishing a Backbone of the image segmentation and classification network, establishing a feature pyramid network (FPN), and establishing a feature aggregation module (FAM);
S04: model pre-training: training the image recovery network such that the encoder of the image recovery network fully learns the feature of the cardiac fibrosis image; and
S05: model training: after completion of the model pre-training, using the encoder of the image recovery network as the BackBone of the improved Mask R-CNN, and migrating an encoder parameter trained at a first stage to the Mask R-CNN, and performing model training by automatically decreasing a learning rate using ReduceLROnPlateau.

2. The cardiac fibrosis diagnosis model based on multi-task attentional feature fusion according to claim 1, wherein in step S01, the cardiac MR image comprises two different labels: No. 0 label and No. 1 label; the No. 0 label is a background label, and the No. 1 label is a heart label; whether the heart has fibrosis is determined, and marked with 0 and 1, 0 representing normal and 1 representing fibrosis; and the labeled sample data is divided into a training set and a test set.

3. The cardiac fibrosis diagnosis model based on multi-task attentional feature fusion according to claim 2, wherein in step S02, the normalization processing is performed according to (x−μ)/σ, wherein x represents a hounsfield unit (HU) value of a pixel in a cardiac MR image; u represents an average value of HU values of all pixels; σ represents a standard deviation of all the pixels; and for the data enhancement, Gaussian random noise, random contrast enhancement, random mirroring, random horizontal flipping, and random rotation are used.

4. The cardiac fibrosis diagnosis model based on multi-task attentional feature fusion according to claim 1, wherein in step S03, the feature of the cardiac fibrosis image comprises texture and edge information; the convolutional block is specifically composed of a convolutional layer, a LeakyReLU activation function and a BN layer; the convolutional layer is configured to further extract an advanced feature of the cardiac MR image; the LeakyReLU activation function is configured to introduce a nonlinear change such that the cardiac fibrosis diagnosis model is capable of learning and simulating more complicated function mapping; and the BN layer is configured to increase a training speed.

5. The cardiac fibrosis diagnosis model based on multi-task attentional feature fusion according to claim 4, wherein in step S03, the image segmentation and classification network is configured to further refine and upsample a feature map extracted by the encoder to recover a resolution close to a resolution of an original input image; and the ConvTranspose layer is configured to reduce artifacts and blurs in an image using structural information in training data.

6. The cardiac fibrosis diagnosis model based on multi-task attentional feature fusion according to claim 5, wherein in step S03, an overall network structure is improved based on the Mask R-CNN; a first improvement is made on the BackBone of the image segmentation and classification network, which is replaced with an encoding layer of the image recovery network in step S03, and a parameter of the encoding layer is migrated; a cardiac image recovery task is completed by the image recovery network through self-supervised learning; semantic information and a typical feature of the cardiac image are learned by the encoding layer; a network parameter is migrated, and a convergence rate of the improved Mask R-CNN model is increased; a dual-level feature pyramid network structure is introduced into the FPN, and comprises a pixel-FPN and a region-FPN to capture a global context and local details of the cardiac MR image; and the FAM is configured to dynamically adjust a region-feature weight and a pixel-feature weight.

7. The cardiac fibrosis diagnosis model based on multi-task attentional feature fusion according to claim 6, wherein in step S04, in the model training, an Adam optimizer is adopted for the model training, with a specific formula being as follows:
$$MSE = \frac{1}{N} {\sum }_{i = 1}^{N} {( {y}_{i} - {\hat{y}}_{i} )}^{2}$$
wherein N represents a total number of samples; yᵢ represents a real value of an ith sample; and ŷᵢ represents a predicted value of the ith sample.

8. The cardiac fibrosis diagnosis model based on multi-task attentional feature fusion according to claim 7, wherein in step S05, a patience parameter is set for 50 times; when Loss does not decrease in 50 consecutive Epochs, a learning rate automatically decreases to 1/10 of an original learning rate; 5000 rounds of training are performed; a loss function uses a combination of a plurality of losses, comprising a dice loss, a cross entropy loss, and an edge loss proposed for a problem of an unclear boundary of a cardiac fibrosis diseased area; and a formula of the dice loss is as follows:
$${L}_{Dice} = 1 - \frac{2 \times \vert T ⋂ P \vert}{\vert T \vert + \vert P \vert}$$
wherein T represents true labeling; P represents a predicted region; and the dice loss function is configured to maximize an intersection of a model output and a real label.

9. The cardiac fibrosis diagnosis model based on multi-task attentional feature fusion according to claim 8, wherein in step S05, a formula of the cross entropy loss is as follows:
$${L}_{ce} = - [ y log ( \hat{y} ) + ( 1 - y ) log ( 1 - \hat{y} ) ]$$
wherein y represents the real label, and y represents a prediction probability of 0 to 1.

10. The cardiac fibrosis diagnosis model based on multi-task attentional feature fusion according to claim 9, wherein a calculation method of the edge loss designed for the problem of an unclear cardiac fibrosis boundary comprises:
a first step: generating an edge map, and using a laplacian operator to operate a real mask ground-truth mask to obtain a soft edge map reflecting edge information, wherein the laplacian operator is a second order derivative operator and is capable of highlighting an edge and a detail in an image; and specific steps are as follows:
1, defining a laplacian kernel, wherein the kernel used is as follows:
$$K = [ \begin{matrix}0 & 1 & 0 \\ 1 & - 5 & 1 \\ 0 & 1 & 0\end{matrix} ]$$
2, a convolution operation: performing a convolution operation using the laplacian kernel and the real mask; and for each pixel in the real mask, multiplying a pixel in a neighborhood thereof by a corresponding value in the laplacian kernel, and then summating results to obtain a new value of the pixel point; and
3, result conversion, superposing an image result after processing with a laplacian filter kernel and an original image to enhance an edge while retaining an image edge content;
a second step: performing thresholding processing on the soft edge map obtained through the laplacian operator, and converting the soft edge map to a binary edge map, wherein in the binaryzation process, pixels in the edge map are divided into two an edge type and a non-edge type;
a third step: predicting an edge map: repeating the first step and the second step with a mask predicted by a model to obtain a predicted edge map; and
a fourth step: calculating an edge loss: calculating the edge loss using the formula of the dice loss, thereby obtaining a training loss function Lₜₒₜₐₗ: Lₜₒₜₐₗ=L_{Dice}+L_{ce}+Ledge.
